(19) United States
(12) Patent Application Publication
(10) Pub. No.: US 2007/0060905 A1
(43) Pub. Date: Mar. 15, 2007

(54) Needle tether

(75) Inventor: Glade H. Howell, Sandy, UT (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(73) Assignee: Becton Dickinson and Company

(21) Appl. No.: 11/204,976

(22) Filed: Aug. 16, 2005

(51) Int. Cl. A61M 31/00

(52) U.S. Cl. 604/500; 604/164.01

(57) ABSTRACT

A new type of tether that may be used in conjunction with a needle and a needle shield is disclosed. The tether is designed such that if a needle is retracted from a human patient, the tether will help to secure the tip of the needle within the needle shield. The tether will generally comprise a body and one or more flaps that are added to the body. Each of the flaps will also include at least one aperture. The apertures and the flaps are designed such that the needle may be threaded through the apertures in the flaps.

### TECHNICAL FIELD

The present invention relates generally to a tether that may be used in conjunction with a needle and a needle shield device. More specifically, the present invention relates to an improved tether having flaps that will retain a needle tip within the needle shield.

### BACKGROUND OF THE INVENTION

A significant concern in modern medicine is the occurrence of unintentional punctures or pricks with medical needles (sometimes referred to as “needlesticks”). Needles are of course widely used in most types of medical procedures. Needles are used to deliver and extract fluids such as pharmaceuticals and body fluids, notably blood. Needles are also used to establish vascular and arterial access. For example, needles are used to insert catheters into a vein or artery so that access to that vein or artery can be had over the necessary period of time. Intravenous fluids can then be introduced into the body through a line attached to the catheter.

Catheters are used for infusing fluid, such as normal saline solution, various medicaments, and total parenteral nutrition, into a patient, withdrawing blood from a patient, or monitoring various parameters of the patient's vascular system. A common type of intravenous (IV) catheter is an over-the-needle peripheral IV catheter. As its name implies, an over-the-needle catheter is mounted over an introducer needle having a sharp distal tip. At least the distal portion of the catheter tightly engages the outer surface of the needle to prevent peelback of the catheter and thus facilitate insertion of the catheter into the blood vessel. The catheter and the introducer needle are assembled so that the distal tip of the introducer needle extends beyond the distal tip of the catheter with the bevel of the needle facing up away from the patient's skin. The catheter and introducer needle are generally inserted at a shallow angle through the patient's skin into a blood vessel.

In order to verify proper placement of the catheter in the blood vessel, the clinician generally confirms that there is “flashback” of blood in a flashback chamber of the catheter assembly. Once proper placement of the catheter into the blood vessel is confirmed, the clinician applies pressure to the blood vessel by pressing down on the patient's skin over the blood vessel distal of the introducer needle and the catheter. This finger pressure occludes the vessel, minimizing further blood flow through the introducer needle and the catheter. The clinician then withdraws the introducer needle, leaving the catheter in place, and attaches an appropriate device to the catheter. In traditional practice, the completion of this process left an exposed needle, which was at the same time blood contaminated.

With such wide use of needles leaving exposed needles following the subject procedure, it is not surprising that a common injury to medical personnel involves the inadvertent puncture with a needle. “Needlesticks” are typically the most common type of accidental injury to medical personnel. Needlesticks range from painful annoyances to serious injuries. In contemporary medical practice these injuries have become a more serious threat with the increasing presence of blood-borne diseases.

The Federal Government and certain states have adopted, or are considering adopting, rules and regulations governing the use of needles. Such rules and regulations commonly include a provision that requires that the needle-employing device include some mechanism for blunting the needle, retracting the needle into a safe container, or other manner of preventing the needle from causing an inadvertent needlestick once it has served its purpose. With these factors in mind, several inventors and medical device manufacturers have been active in developing needleless access devices, self-blunting needle devices, and catheter devices which include some type of containment mechanism for the used needle.

“Needle shields” or needle-shielding devices are devices that have been designed in order to comply with these new governmental standards/regulations. In general, a needle shield is a housing, sleeve, or other similar device that is designed such that when the needle is withdrawn from the patient, the needle tip will be trapped/captured within the needle shield. The purpose of these needle shield devices is to house the tip of the needle in a secure location, thereby avoiding the possibility of needlesticks.

Typically, the needle shield will operate through the use of tethers and tether systems. In general, these tether systems are of a known length and are attached to both the needle (via the needle handle or hub) and the needle shield. These tether systems are designed to restrain the movement of the needle and to further hold the needle tip within the needle shield. More specifically, the tether system is designed such that if the user attempts to pull/retract the needle tip from the needle shield, the tether will prevent such movement and will hold the needle tip within the interior of the needle shield.

Of course, in order for the tether to adequately hold the needle tip within the needle shield, the exact length of the tether must be established with precision and accuracy. More specifically, the length of the tether must be set such that even if the tether is fully extended, the tether will still be able to hold the needle tip within the interior of the needle shield.

Unfortunately, many known tether systems are designed such that the needle is woven/threaded through a portion of the tether. While such threading is an effective means of attaching the tether to the needle, this type of weaving often means that the tether may be able to stretch during use. Such stretching of the tether is disadvantageous in that it introduces tolerance/slack into the length of the tether.

Accordingly, there is a need in the art for a new type of tether that will eliminate the possibility that the tether will stretch during use. Such a tether is disclosed herein.

### BRIEF SUMMARY OF THE INVENTION

In order to overcome the limitations discussed above, the present invention relates to a new type of tether that may be used in conjunction with a needle and a needle shield. As is known in the art, a needle shield is a device that is designed to capture/retain a needle after the needle has been inserted into a patient. The tether of the present invention is designed to be used in conjunction with the needle/needle shield and is designed to prevent the needle tip from being withdrawn from the needle shield.

Like other known tethers, the tether of the present invention is designed such that the tip of the needle will be threaded through openings in the tether. The tether is designed such that when the tether is in the fully extended position, the tip of the needle will be positioned within the needle shield. Accordingly, if the user attempts to retract/pull the needle tip out of the needle shield, the tether will resist such movement and will hold the needle tip within the needle shield.

The tether generally comprises a body. This body is generally a rectangular piece of material. Usually, the material used to make the tether will be a non-stretch material. The body will have a proximal end and a distal end. The proximal end is designed such that it may be attached to the needle hub whereas the distal end is designed such that it may be attached to the needle shield.

The body may additionally comprise one or more pleats. The tether is designed such that it may be folded along the pleats. More specifically, the tether is designed such that prior to insertion of the needle into the patient, the tether will be folded along pleats into a generally accordion shape. Of course, when the tether is pulled into the extended position, the tether will generally unfold into a long, rectangular-shaped member.

Unlike some of the previously known tethers, the tether will also include one or more flaps. These flaps are usually cut or punched out of the tether using a die cut machine or other similar device. Of course, other embodiments may be made in which the flaps are added and/or attached to the body of the tether.

The flaps will usually be perpendicular or generally perpendicular to the longitudinal direction of the tether. Each of the flaps will also include one or more apertures. In general, these apertures are designed such that the needle may be threaded through the apertures in the flaps. Thus, instead of having the needle threaded through openings in the center of the tether, as is known in the art, the present embodiments will have the needle threaded through the apertures in the flaps.

Threading the needle through apertures in the flaps may provide significant advantages over the prior art systems that have the needle threaded through openings in the center of the tether. More particularly, one of the drawbacks of previously known systems is that by threading the needle through openings in the center of the tether (as is done in these previously known systems), the tether may be able to expand/stretch. Again, this type of stretching of the tether is disadvantageous in that it introduces tolerance/uncertainty into the length of the expanded tether. However, by threading the needle through apertures in the flaps, the possibility of the tether stretching is eliminated and this problem of the prior art is effectively overcome.

### BRIEF DESCRIPTION OF THE SEVERAL VIEWS OF THE DRAWINGS

In order that the manner in which the above-recited and other features and advantages of the invention are obtained will be readily understood, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 is perspective view of a catheter, a needle and needle shield that are used in conjunction with an embodiment of a tether according to the present invention; and

FIG. 2 is perspective view of the embodiment of FIG. 1 which shows the way in which the tether may engage the needle.

### DETAILED DESCRIPTION OF THE INVENTION

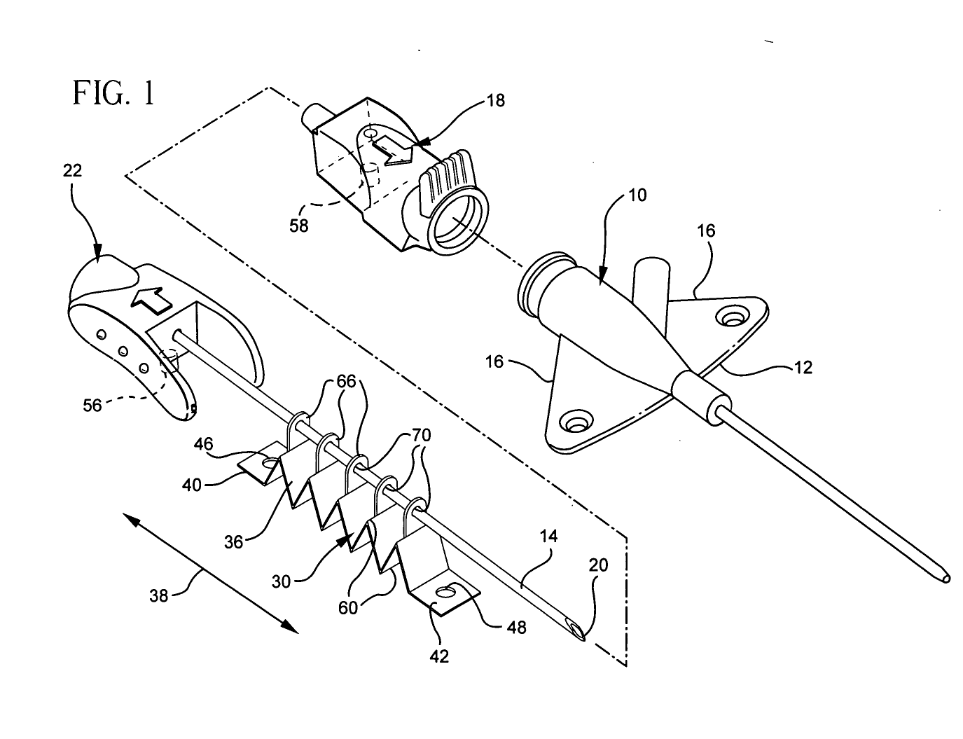

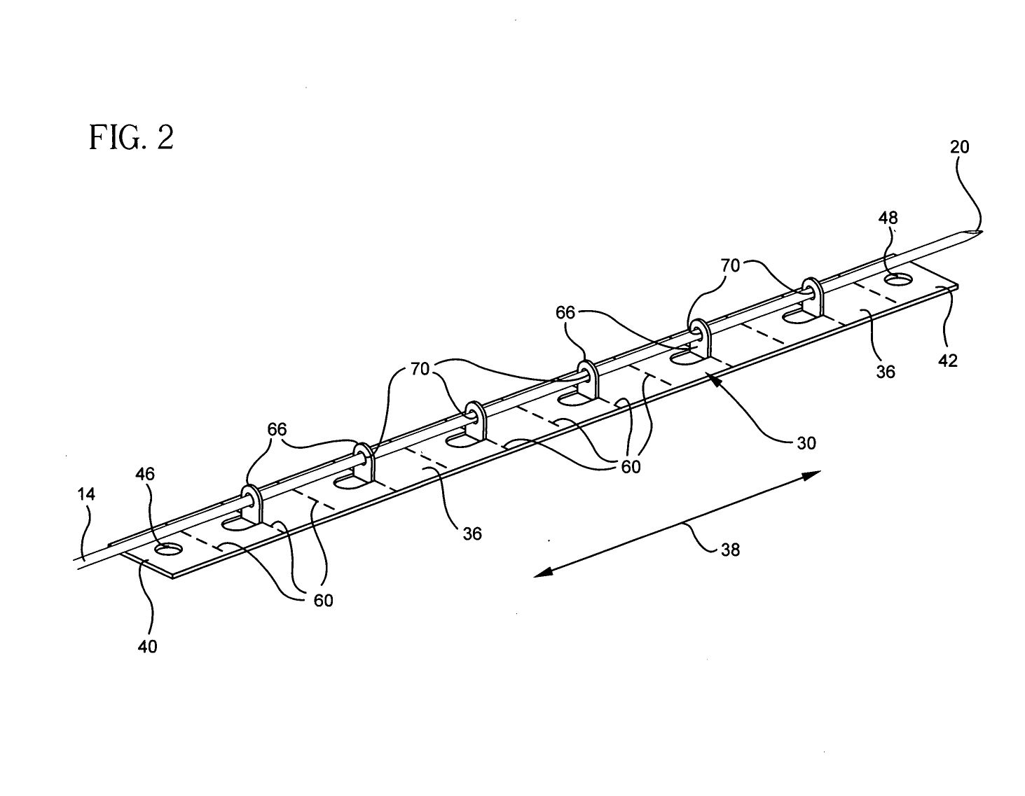

The presently preferred embodiments of the present invention will be best understood by reference to the drawings, wherein like parts are designated by like numerals throughout. It will be readily understood that the components of the present invention, as generally described and illustrated in the figures herein, could be arranged and designed in a wide variety of different configurations. Thus, the following more detailed description of the embodiments of the tether of the present invention, as represented in FIGS. 1 through 2 is not intended to limit the scope of the invention, as claimed, but is merely representative of presently preferred embodiments of the invention.

FIG. 1 is a perspective view of a catheter assembly 10 and a needle 14. Both the catheter assembly 10 and the needle 14 are known in the art and are used to introduce fluid into human patients. The needle 14 has a needle tip 20 and a needle hub 22 (which is sometimes called the needle handle). The needle 14 and the catheter assembly 10 are similar and/or identical to the devices that are shown and described in shown in U.S. Pat. Nos. 6,606,181 and 6,171,287 (which patents are expressly incorporated herein by reference). Accordingly, for more information regarding the catheter assembly 10 and the needle 14, the reader should consult these patents and the other patents cited herein. Of course, those of skill in the art will recognize that other types of devices may also be used as the catheter assembly 10 or the needle 14.

As can be seen in FIG. 1, the catheter assembly 10 is essentially a standard over-the-needle catheter. In the fully assembled configuration, the distal tip 20 of the needle 14 extends from the distal end of the catheter assembly 10. As mentioned above, this is the standard placement of an over-the-needle catheter widely used in the art.

The catheter assembly 10 also includes a catheter adapter 12 attached to a needle shield 18. The catheter adapter 10 is of the type known in the art. The catheter adapter 12 includes wings 16. The wings 16 facilitate manipulation and placement of the catheter assembly 10. The wings 16 can also be used to secure the catheter assembly 10 to the patient once the catheter assembly 10 has been inserted, by providing a surface for tape or the like.

In the embodiment shown in FIG. 1, a needle shield 18 is also illustrated. As is known in the art, a “needle shield” is a device that is designed such that when the needle 14 is withdrawn from the patient, the needle shield 18 will capture and retain the tip 20 of the needle 14 within the needle shield 18. Such retention of the needle tip 20 is important in that it prevents (as described above) the needle tip 20 from accidentally impaling or injecting the clinician.

Again those of skill in the art will recognize that a variety of different types of devices may be used as the needle shield 18. In fact, any device that is capable of retaining the needle tip 20 after it has been withdrawn from the patient may be used as the needle shield 18. The particular example of the needle shield 18 in FIG. 1 is a housing through which the needle 14 may pass. Other types of needle shields and/or needle-shielding devices may also be used.

As shown in FIG. 1, a tether 30 is also illustrated. The tether 30 is designed to be used in conjunction with the needle 14 and the needle shield 18. Like other tethers that are known in the art, the tether 30 is designed such that it may be used to help retain the needle tip 20 within the needle shield 18. Accordingly, additional information regarding tethers and the way in which they may be used with needles/needle shields may be found in U.S. Pat. No 6,527,747 (which patent is expressly incorporated herein by reference and will be referred to herein as the “'747 patent”).

A variety of different materials may be used to construct the tether 30 according to the present invention. In some of the presently preferred embodiments, the material used to make the tether 30 is a “non-stretch” material such as polyethylene terephthalate. A “non-stretch” material is any material that is stiff or relatively stiff material that will not expand or stretch when the tether 30 is expanded into the extended position. Generally, these “non-stretch” materials will be plastics or other similar materials. Of course, those of skill in the art will recognize that a variety of other types of materials may also be used to construct the tether 30, including materials that may stretch or expand when the tether 30 is expanded into the extended position. However, the use of such “stretching” materials is not presently preferred.

As can be seen in FIG. 1, the tether 30 generally comprises a body 36. The body 36 may be a rectangular piece of material that is configured such that the longitudinal direction 38 of the body 36 is parallel to the needle 14. Of course, other shapes and/or configurations may also be used.

In general, the body 36 will have a proximal end 40 and a distal end 42. The proximal end 40 is designed such that it may be attached to the needle hub 22 whereas the distal end 42 is designed such that it may be attached to the needle shield 18. In the embodiment shown in FIG. 1, such attachment is accomplished using a proximal hole 46 and a distal hole 48. The proximal hole 46 is added to the proximal end 40 whereas the distal hole 48 is added to the distal end 42. The holes 46, 48 are sized and configured such that they may engage the attachment prongs 56, 58 that are added to the needle hub 22 and the needle shield 18. More specifically, the prong 56 is designed such that it may pass through the hole 46 (as is illustrated by the dashed lines) and connect the proximal end 40 to the needle 14 and/or the needle hub 22. Likewise, the prong 58 is designed such that it may pass through the hole 48 (as is illustrated by the dashed lines) and connect the distal end 42 to the needle shield 18.

The holes 46, 48 are an inexpensive and effective mechanism for attaching the tether 30 to the needle 14 and the needle shield 18. Accordingly, the use of such holes 46, 48 is one of the presently preferred methods for attaching the ends 40, 42 to the needle 14 and the needle shield 18. Those of skill in the art will recognize, however, that other methods and/or mechanisms for attaching the ends 40, 42 and/or the tether 30 to the needle 14/needle shield 18 may also be used.

The body 36 may additionally comprise one or more pleats 60. (The pleats are also illustrated with clarity in FIG. 2). The tether 30 is designed such that it may be folded along the pleats 60. More specifically, the tether 30 is designed such that prior to the time that the needle 14 is inserted into the patient, the tether 30 will be folded along pleats 60 into a generally accordion shape (as shown in FIG. 1).

As shown in FIG. 1, the tether 30 is positioned along the longitudinal direction 38 of the needle 14 such that it is positioned between the hub 22 and the needle shield 18. However, the length of the tether 30 is less than the distance between the needle hub 22 and the needle shield 18. When the needle 14 is passed through the needle shield 18 and the catheter assembly 10, the body 36 of the tether 30 will be in a compacted, accordion-folded position between the needle shield 18 and the hub 22. Of course, as noted above, when the tether 30 is in this configuration, the needle tip 20 will extend out of the distal end of the catheter assembly 10.

After the catheter assembly 10 has been inserted into the patient, the clinician will withdraw the needle 14 from the patient. As noted above, such withdrawal of the needle 14 will position the needle tip 20 on the interior of the needle shield 18. As described above, the needle shield 18 will have a locking mechanism (not shown) which prevents the needle 14 from being re-extended out of the needle shield 18 towards the catheter assembly 10.

The withdrawal of the needle tip 20 into the interior of the needle shield 18 will also affect the tether 30. As noted above, the tether 30 is attached to the needle hub 20. Accordingly, as the needle 14 is moved, such movement will also affect the tether 30. More specifically, as the needle 14 is moved and the needle tip 20 is withdrawn, the tether 30 will expand/extend in the longitudinal direction 38. (This movement of the tether 30 will not cause significant stretching of the tether 30 because the tether 30 is made of a non-stretch material.) In turn, this expansion/extension of the tether 30 means that, once the needle tip 20 has been withdrawn into the needle shield 18, the tether 30 is no longer in the compacted/accordion-folded configuration.

The tether 30 is configured such that it will prevent the needle tip 20 from being withdrawn out of the needle shield 18 (in the direction of hub 22). As noted above, the length of the extended tether 30 is less than the distance between the needle hub 22 and the needle shield 18. Thus, when the tether 30 is in the fully extended position, the tip 20 of the needle 14 will be positioned within the needle shield 18. Accordingly, if the user attempts to further retract the needle 14 (i.e., to further pull the needle 14 such that the tip 20 would exit the needle shield 18), the fully extended tether 30 will resist this movement/retraction of the needle 14 and operates to hold the tip 20 within the needle shield 18. In fact, the only way that the user can retract the tip 20 out of the needle shield 18 will be to break the tether 30. Thus, in this manner, the tether 30 holds the needle tip 20 within the needle shield 18 and protects the user from being accidentally impaled/injected with the needle tip 20.

A flap 66 is also added to the tether 30. A flap 66 is a tab or extension of material that is generally cut out from the body 36 (as will be described in greater detail in conjunction with FIG. 2). Other embodiments may also be made in which the flap 66 is attached to the body 36.

The flap 66 will generally be made of the same material that is used to make the body 36. As shown in FIG. 1, more than one flap 66 may also be used. The exact number of flaps 66 will depend upon the length of the tether 30, the length of the needle 14, and/or other factors known to those of skill in the art.

As will be described in greater detail below, the flaps 66 are cut out from the body 36. Accordingly, when the tether 30 is in the compacted/accordion-folded position, the flaps 66 will be perpendicular or generally perpendicular to the longitudinal direction 38. However, other orientations/configurations of the flaps 66 may also be used.

In the shown embodiment, each of the flaps 66 will also include at least one aperture 70. An aperture 70 is an opening or hole that is located on the flap 66. The apertures 70 are usually positioned proximate to the middle of the flaps 66. However, other positions may also be used. The apertures 70 are also designed such that the needle 14 may be threaded through the apertures 70. Thus, the apertures 70 may have a rounded shape and a diameter that is substantially equal to and/or slightly larger than the diameter of the needle 14. Other sizes and/or configurations for the apertures 70 may also be used.

It should also be noted that, in other embodiments, each of the flaps 66 will not comprise an aperture 70. For example, embodiments may be made in which there are multiple flaps 66 and only one (or a select few) of the flaps 66 comprise an aperture 70. Still other embodiments may be constructed in which every other flap 66 (or every third flap, every fourth flap, etc.) comprises an aperture 70. Those of skill in the art will recognize that a variety of different configurations regarding the number of apertures 70 and/or the number of flaps 66 may be used. Yet additional embodiments may be constructed in which the tether 30 only has one flap 66 and that single flap comprises and aperture 70. Yet additional embodiments may be constructed in which one or more of the flaps 66 comprise a plurality of apertures 70.

In the embodiment shown in FIG. 1, the size of the apertures 70 is smaller than that which is normally found on tethers known in the art. The tether 30 may be narrower that other tethers due to this reduction in size of the aperture 70. In some embodiments, such a reduction in the size of the apertures 70 may operate to reduce the overall stretching of the tether 30. However, other embodiments may also be made in which the apertures 70 are the same size as and/or larger than the holes/openings known in the art.

As noted above, the flaps 66 and the apertures 70 are designed such that the needle 14 may be threaded through the apertures 70 that are positioned on the flaps 66. Such threading of the needle 14 through the apertures 70 in the flaps 66 may, in some embodiments, provide significant advantages. As illustrated in the '747 patent, many of the currently known tethers have the needle thread through large openings (in a common “over-and-under” pattern) that are located in the center of the tether. The drawback with threading the needle through center openings is that this configuration will allow the tether to stretch when the tether is expanded. Such stretching may be disadvantageous in that it makes it difficult to determine the exact length of the extended tether. Accordingly, if the tether stretches “too much,” the tether may not be capable of adequately holding the needle tip within the needle shield.

However, the tether 30 of the present embodiments overcomes these limitations. For example, the tether 30 is made of a non-stretch material which eliminates/reduces the potential stretching of the tether 30. More importantly, the use of the flaps 66 also help to eliminate any potential for stretching of the tether 30. By using the flaps 66 and the apertures 70 as the mechanism for attaching the needle 14 to the tether 30, the unwanted stretching of the tether 30 will effectively be eliminated. More particularly, the use of the flaps 66 means that the extended length of the tether 30 will be known with precision and accuracy. As a result, the possibility that the tether 30 will stretch “too much” and allow the needle tip 20 to be withdrawn out of the needle shield 18 is drastically reduced and/or eliminated. Likewise, because the length of the tether 30 will be known with precision and accuracy, shorter, smaller and/or more compact needle shields 18 may be used.

Referring now to FIG. 2, a perspective view of the embodiment of the tether 30 of FIG. 1 illustrates the way in which the tether 30 may engage the needle 14. Specifically, FIG. 2 shows the tether 30 is the extended position, such as would occur when the needle 14 is retracted from the catheter assembly 10. For purposes of clarity and illustration however, the needle shield 18 (which would normally be present in the system to retain the needle tip 20) is not illustrated.

In the exemplary embodiment shown in FIG. 2, the length of the tether 30 (in the extended position) may be between about 6 to 7 centimeters. Other embodiments may use different lengths and sizes of the tether 30 may also be used, which will depend upon factors such as the length of the needle, the length of the tether, the strength of the tether, etc. The pleats 60 may be positioned along the longitudinal direction 38 of the tether 30. In some exemplary embodiments, the pleats 60 will be evenly spaced across the tether 30 such that the distance between any two adjacent pleats 60 may be approximately 5 millimeters. Of course, other sizes and/or distances between the pleats may also be used. Additional embodiments may have the pleats 60 arranged differently and/or have different lengths/distances between the pleats 60 based upon factors such as the shape of the tether 30, the length of the tether 30, the length of the needle, the strength of the tether 30, the total number of pleats 60, etc.

As shown in FIG. 2, the flaps 66 may be formed directly from the material that is used to form the tether 30. In the embodiments shown in FIG. 1, this is accomplished by cutting the flaps 66 (using a “die” cut machine or other similar device) directly into the tether 30. Once the flaps 66 have been cut into the tether 30, the flaps 66 may be, as shown in FIG. 2, raised up so that they are generally perpendicular to the longitudinal direction 38. Of course, those of skill in the art will recognize that other methods for forming and/or attaching the flaps 66 to the body 36 may also be used, including welding, weaving, stitching, etc.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. A tether for use with a needle and a needle shield, the tether comprising:
a body having a proximal end and a distal end, wherein the proximal end is attachable to a needle hub and the distal end is attachable to a needle shield;
a flap attached to and extending from the body, the flap comprising an aperture designed such that the needle may be threaded through the aperture.

2. A tether as in claim 1 wherein the tether is configured such that when the tether is extended, the tether will hold the tip of the needle within the needle shield.

3. A tether as in claim 1 wherein tether comprises a plurality of flaps, wherein the flaps are cut from the body of the tether.

4. A tether as in claim 3 wherein the flaps are configured such that when the needle is threaded through the aperture, the flap is generally perpendicular to the longitudinal direction of the tether.

5. A tether as in claim 1 wherein the tether is folded along pleats in the body such that prior to insertion of the needle into a patient, the tether will have a generally accordion shape.

6. A tether as in claim 1 wherein the tether is made of a non-stretch material.

7. A tether as in claim 1 wherein the aperture is substantially equal in diameter to the diameter of the needle.

8. A tether as in claim 1 wherein the aperture is slightly larger in diameter to the diameter of the needle.

9. A tether as in claim 1 wherein the tether comprises a plurality of flaps and wherein each flap has an aperture.

10. A method for retaining a needle tip within a needle shield using a tether, the method comprising:
obtaining a tether comprising a body and a flap, wherein the body has a proximal end and a distal end and the flap comprising an aperture;
threading the needle through the aperture; and
attaching the proximal end to a needle hub and the distal end to the needle shield, wherein the length of the tether is less than the distance between the needle hub and the needle tip such that the tether prevents the user from withdrawing the needle tip out of the needle shield.

11. The method of claim 10 further comprising the step of folding the tether into an according shape.

12. The method of claim 10 wherein the attaching step is performed after the threading step.

13. The method as in claim 10 wherein the tether is made of a non-stretch material.

14. A tether for use with a needle and a needle shield, the tether comprising:
a body means having a proximal end and a distal end, wherein the proximal end is attachable to a needle and the distal end is attachable to a needle shield;
an attachment means for attaching the tether to the needle, the attachment means being attached to the body, wherein the attachment means is designed to engage the needle.

15. A tether as in claim 14 wherein the tether is configured such that when the tether is extended, the tether will hold the tip of the needle within the needle shield.

16. A tether as in claim 14 wherein attachment means comprises at least one flap, wherein the flap is cut from the body of the tether, and wherein the at least one flap comprises an aperture.

17. A tether as in claim 16 wherein the flap is configured such that when the needle is threaded through the apertures, the flap is generally perpendicular to the longitudinal direction of the tether.

18. A tether as in claim 14 wherein the tether is folded along pleats in the body such that prior to insertion of the needle into a patient, the tether will have a generally accordion shape.

19. A tether as in claim 14 wherein the tether is made of a non-stretch material.

20. A tether as in claim 14 wherein the attachment means is a plurality of flaps and wherein each flap has an aperture.

21. A tether as in claim 14 wherein the attachment means further comprises an aperture that is substantially equal or slightly larger in diameter to the diameter of the needle.
